(19) United States
(12) Patent Application Publication
(10) Pub. No.: US 2007/0055164 A1
(43) Pub. Date: Mar. 8, 2007

(54) Physiological status monitoring system and method

(75) Inventors: Hung-Jen Huang, Rende Shiang (TW); Shao-Wei Chung, Zhudong Town (TW); Chie-Chung Kuo, Keelung City (TW); Hsin-Teng Lin, Huwei Town (TW)

Correspondence Address: BRUCE H. TROXELL, SUITE 1404, 5205 LEESBURG PIKE, FALLS CHURCH, VA, 22041, US

(73) Assignee: Industrial Technology Research Institute

(21) Appl. No.: 11/266,276

(22) Filed: Nov. 4, 2005

(30) Foreign Application Priority Data
Sep. 2, 2005 (TW) 094130041

(51) Int. Cl. A61B 5/02; G08B 23/00

(52) U.S. Cl. 600/508; 340/575; 340/576

(57) ABSTRACT

A physiological status monitoring method is disclosed, which comprises the steps of: monitoring physiological status of at least an individual stationed inside a transportation means for acquiring a physiological data of the individual; transmitting the physiological data to a center processing device; and processing an operation of analysis and comparison upon the received physiological data by the center processing device. Moreover, with respect to the aforesaid method, a physiological status monitoring system can be provided to be installed inside a transportation means, which comprises: at least a bio sensor, capable of monitoring and sensing a physiological status of at least an individual stationed inside the transportation means for generating a sensing signal; and a center processing device, arranged inside the transportation means for receiving the sensing signal and thus evaluating the physiological caodition of the monitored individual inside the transportation means. Since the physiological status of the individual stationed inside a transportation means can be monitored by the physiological status monitoring system provided by the present invention, the happening of traffic accidents can be minimized.

### FIELD OF THE INVENTION

The present invention relates to a physiological status monitoring system and method, and more particularly, to a system and method capable of monitoring individuals stationed inside a transportation means for acquiring physiological data of the same, such as the rate of heat beats and pulses, while using the acquired physiological data as basis for determining the mental status of those individuals so as to issue a alarm while detecting abnormalities.

### BACKGROUND OF THE INVENTION

In the modern and industrialized society, the percentage of households with more than one car is increasing. However, the likelihood of traffic accident will also increase with the increase of cars. Based on traffic accident data, most accidents are caused by human errors, such as driving wile intoxicated (DWI), speeding or having a heart attack or dozing off.

It is noted that the happening of physical problem during driving, such as heart attack or drowsiness, usually is unexpected and thus is difficult to prevent. For instance, a driver might be in high spirit at the starting of a long distance drive, but is fallen into a doze after driving for a period of time without stopping, in particular, when he is driving along and there is no one to remind he of the situation, that it is usually a case of traffic accident.

The means commonly adopted by driver monitoring systems of prior arts are as following:
1. Image matching infrastructure: the means of image matching infrastructure uses an image fetch device such as camera to acquire images of a driver-under-monitoring and then compare the acquired images with certain standard images stored in the database of the infrastructure so as to determine the physical condition of the driver-under-monitoring.
2. Infrastructure of eye movement analysis: the means of eye movement infrastructure detect the rate of eye blinking and the information of staring direction of a driver-under-monitoring, and then compare the detected information with the database of the infrastructure so as to determine the mental condition of the driver-under-monitoring.
3. Infrastructure of head movement analysis: the means of head movement infrastructure detect and evaluate the positions pinpointed by a head movement and the magnitude of the same of a driver-under-monitoring so as to identified the driving of the driver-under-monitoring.
   Nevertheless, the aforesaid means of prior-art driver monitoring systems will waste a great many of its resource in building a large database since there will be many standard images required to be stored therein. In addition, The infrastructure using image processing for detection and evaluation might not be accurate enough so that the driver monitoring system applying the same night not be able to function normally.

Therefore, it is in great need to have a system and method capable of monitoring individuals stationed inside a transportation means for acquiring physiological data of the same while using the acquired physiological data as basis for determining the driving condition of those individuals.

### SUMMARY OF THE INVENTION

In view of the disadvantages of prior art, the primary object of the present invention is to provide a system and method capable of monitoring individuals stationed inside a transportation means for acquiring physiological data of the same, such as the rates of heat beats, respiration and pulse, while using the acquired physiological data as basis for determining the driving condition of those individuals so as to issue a alarm in time while detecting abnormalities.

Another object of the invention is to provide a physiological status monitoring system and method with low power consumption, enhanced signal penetration and simplified infrastructure, which is realized by the use of a means of ultra wideband (UWB) for acquiring the physiological data of at least an individual stationed inside a transportation means.

It is yet another object of the invention to provide a physiological status monitoring system and method, which is capable of remote monitoring the specific condition of an individual by the use of a mechanism of global communication for transmitting the physiological data of the monitored individual to a monitor center.

To achieve the above objects, the present invention provides a physiological status monitoring method, comprising the steps of: applying a means of ultra wideband for monitoring the physiological status and thus acquiring the physiological data of at least an individual stationed inside a transportation means; transmitting the acquired physiological data to a center processing device; enabling the center processing device to analyze and evaluate whether the received physiological data is normal or not.

In a preferred embodiment of the invention, the physiological status monitoring method further comprises the steps of: enabling an alarm unit arranged in the center processing device of the transportation means to initiate an alerting process while the evaluation performed by the center processing device indicates that the received physiological data is not normal.

Preferably, the physiological status monitoring method further comprises the steps of: enabling a radio frequency transmitter arranged in the center processing device to initiate a process of transmitting the result of the evaluation to a monitor center.

Moreover, to achieve the above objects, the present invention provides a physiological status monitoring system, being arranged in a transportation means, comprising: at least a bio sensor, capable of monitoring and sensing a physiological status of at least an individual stationed inside the transportation means for generating a sensing signal; and a center processing device, arranged inside the transportation means for receiving the sensing signal and thus evaluating the physiological condition of the monitored individual stationed inside the transportation means.

In a preferred embodiment of the invention, the physiological status monitoring system further comprises an alarm unit, being arranged inside the transportation means while coupling to the center processing device, capable of initiating an alerting process with respect to the signal received from the center processing device.

Preferably, the physiological status monitoring system further comprises a radio frequency transmitter, being arranged inside the transportation means while coupling to the center processing device, for transmitting a signal to a monitor center with respect to the signal received from the center processing device. It is noted that the radio frequency transmitter can be a device of global system for mobile communications.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a flow chart depicting a physiological status monitoring method according to the present invention.

FIG. 2A is a schematic view of a physiological status monitoring system according to a preferred embodiment of the invention.

FIG. 2B is a schematic diagram showing the operation of a physiological status monitoring system according to the invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

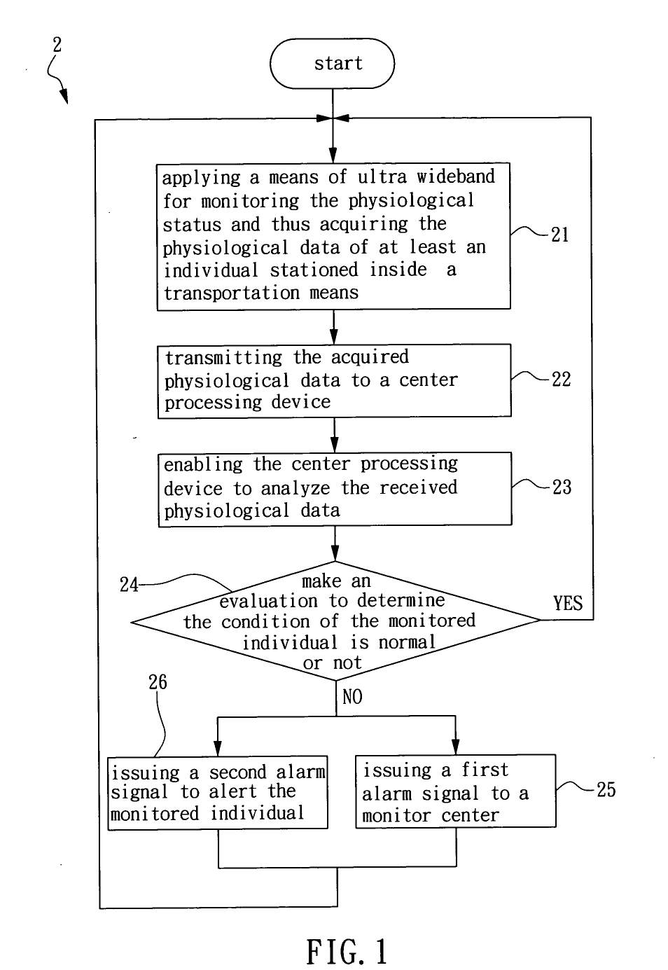

Please refer to FIG. 1, which is a flow chart depicting a physiological status monitoring method according to the present invention. The flow chart of FIG. 1 is used to monitor the physiological statuses of individuals stationed inside a transportation mean, whereas the transportation means is generally referring to those wheeled vehicles, such as truck, touring bus and coach, etc., however, it can also be referred to aircrafts which have persons stationed therein. In addition, the physiological status monitoring method can be used to monitor no only the driver of the transportation means, but also the passengers thereof can be monitored, which includes the patient riding an ambulance a, the passengers of old age riding an aircraft or touring bus, and so on.

As seen in FIG. 1, the physiological status monitoring method comprises the steps of:
step 21: applying a means of ultra wideband for monitoring a physiological status and thus acquiring the data of the physiological status of at least an individual stationed inside a transportation means;
step 22: transmitting the acquired physiological data to a center processing device;
step 23: enabling the center processing device to analyze the received physiological data;
step 24: make an evaluation to determine whether the condition of the monitored individual is normal or not;
step 25: issuing a first alarm signal to a monitor center if abnormal;
step 26: issuing a second alarm signal to alert the monitored individual if abnormal.

It is noted that the physiological status referred in the method of the invention includes heat beat, respiration, and pulse, etc. Accordingly, a database can be built previously into the center processing device of step 22, which can be used as the standard for evaluation the physical condition of the monitored individual. In a preferred embodiment of the invention, the database might comprises the information of the rate of heat beat, respiration, or pulse while the monitored individual is dozing, and the information of the relationship between the rate of heat beat, respiration, or pulse and the physical condition of the monitored individual. Moreover, the transmitting of the first alarm signal to the monitor center of step 25 can be perform by way of a means of global system for mobile communication so that the transmission can be carried out in long range.

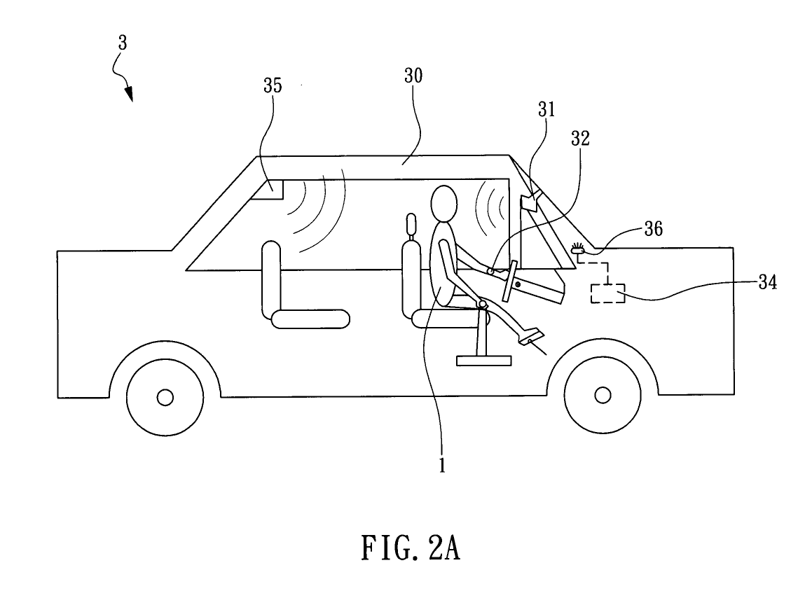

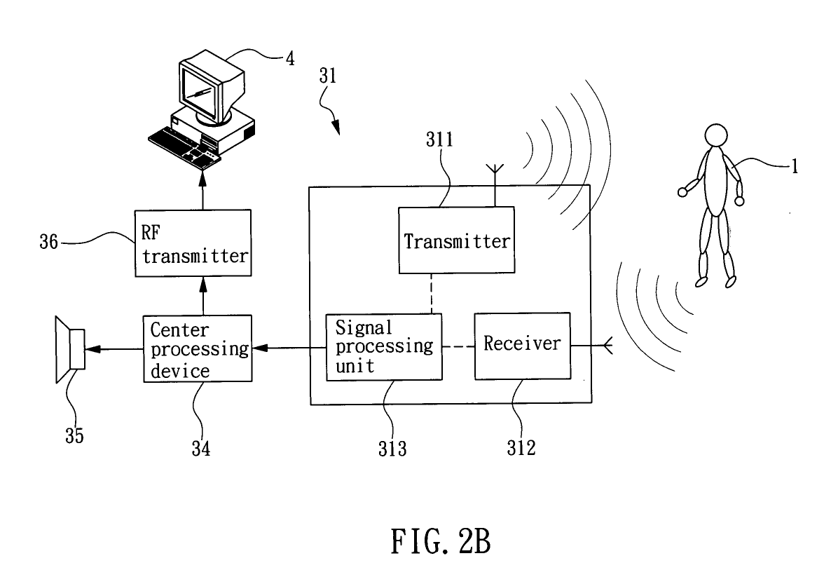

Please refer to FIG. 2A, which is a schematic view of a physiological status monitoring system according to a preferred embodiment of the invention. As seen in FIG. 2A, a physiological status monitoring system is arranged inside a transportation means 30, which is a car in this preferred embodiment, and thus the physiological status monitoring system can be used to monitor the physical condition of an individual 1, which is the driver, so as to prevent traffic accident caused by the poor condition of the driver from happening. The physiological status monitoring system of the preferred embodiment comprises: two bio sensors 31, 32, each capable of monitoring and sensing a physiological status of the driver 1 stationed inside the car 30 for generating a sensing signal; and a center processing device 34, arranged inside the car 30 for receiving the sensing signal and thus evaluating the physiological condition of the driver 1.

It is noted that the amount of the bio sensor is related to the amount of individuals stationed inside the transportation means 30 that require to be monitored. In addition, the physiological status monitoring system further comprises an alarm unit 35, being arranged inside the transportation means 30 while coupling to the center processing device 34, which is capable of initiating an alerting process with respect to the signal received from the center processing device 34. Moreover, a radio frequency transmitter 36 is further being arranged inside the transportation means while coupling to the center processing device 34, which is used for transmitting a signal to a monitor center with respect to the signal received from the center processing device 34. It is noted that the radio frequency transmitter can be a device of global system for mobile communications.

Any bio sensor used in the physiological status monitoring system of the invention can be either a contact-type device or a non-contact device. The bio sensor 32 of FIG. 2 is a contact-type device capable of being worn on the wrist of the driver 1, that is only one of the embodiment and is not limited thereby. The non-contact bio sensor 31 of FIG. 2 can be arranged at a position inside the transportation means 30 which is proper to detect the physiological status of the driver 1. The bio sensor of the present invention can be a device capable of emitting signals of ultra wideband (UWB), which can detect and evaluate the variation of the status of an object-under-monitor within a specific distance with respect to the variation of the reflected UWB signals. The usage of UWB signals for monitoring a specific variation is similar to those disclosed in U.S. Pat. No. 5,361,070. In the preferred embodiment shown in FIG. 2B, the bio sensor 31 transmits signals to the center processing unit 34 by a wired means while the bio sensor 32 transmits signals to the center processing unit 34 by a wireless means.

Please refer to FIG. 2B, which is a schematic diagram showing the operation of a physiological status monitoring system according to the invention. In this preferred embodiment of FIG. 2B, the bio sensor 31 is disposed on a bracelet worn on the wrist of the driver 1 of FIG. 2A, which comprises a transmitter 311, a receiver 312 and a signal processing unit 313. The transmitter 311 can emit a UWB signal to detect the pulse of the driver 1, that is, as the UWB signal emitted from the transmitter 311 incident to the wrist of the driver 1, the UWB signal will be reflected and the reflected signal will vary with respect to the pulse which can be measure on the wrist. Thereafter, the reflected signal is received by the receiver 312 where it is further being transmitted to the signal processing unit 313 for the process of filtering, amplifying, etc., and then, the filtered/amplified signal is transmitted to the center processing device 34 for evaluation.

As soon as the center processing device 34 receives the signal transmitted from the bio sensor 31, it will first convert the received analog signal into a digital data and then compare the digital data to the standard data stored in the database thereof for detecting abnormality. While an abnormality is detected, the center processing device 34 will enable the alarm unit 35 to issue an alerting signal of notification and also transmit an abnormal signal to the monitor center 4.

If the physiological status monitoring system of the invention is being applied to a ship company, either the headquarter or the division branch of the shipping company can use the monitor center 4 to realize its drivers' condition in real time. If the physiological status monitoring system of the invention is being applied to an emergency center, doctors or other relative personnel can use the monitor center 4 to monitor and master the condition of a patient being transported thereto by an ambulance so that any critical condition happened during the transportation can be monitored and handled in time.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A physiological status monitoring method, comprising the steps of:
applying a means of ultra wideband for monitoring a physiological status and thus acquiring the data of the physiological status of at least an individual stationed inside a transportation means;
transmitting the acquired physiological data to a center processing device; and
enabling the center processing device to perform an evaluate to determine whether the received physiological data is normal.

2. The method of claim 1, further comprising:
enabling an alarm unit arranged in the center processing device of the transportation means to initiate an alerting process while the result of the evaluation indicates abnormal.

3. The method of claim 1, further comprising:
enabling a radio frequency transmitter arranged in the center processing device to initiate a process of transmitting the result of the evaluation to a monitor center.

4. The method of claim 3, wherein the radio frequency transmitter is a device of global system for mobile communications.

5. The method of claim 1, wherein the individual is the driver of the transportation means.

6. The method of claim 1, wherein the individual is a passenger of the transportation means.

7. The method of claim 1, wherein the physiological status is the pulse condition of the monitored individual.

8. The method of claim 1, wherein the physiological status is the heart beat condition of the monitored individual.

9. A physiological status monitoring system, being arranged in a transportation means, comprising:
at least a bio sensor, capable of monitoring and sensing a physiological status of at least an individual stationed inside the transportation means for generating a sensing signal; and
a center processing device, arranged inside the transportation means, for receiving the sensing signal and thus evaluating the physiological condition of the monitored individual stationed inside the transportation means.

10. The system of claim 9, further comprising:
an alarm unit, being arranged inside the transportation means while coupling to the center processing device, capable of initiating an alerting process with respect to the signal received from the center processing device.

11. The system of claim 9, further comprising:
a radio frequency transmitter, being arranged inside the transportation means while coupling to the center processing device, for transmitting a signal to a monitor center with respect to the signal received from the center processing device.

12. The system of claim 11, wherein the radio frequency transmitter is a device of global system for mobile communications.

13. The system of claim 9, wherein the individual is the driver of the transportation means.

14. The system of claim 9, wherein the individual is a passenger of the transportation means.

15. The system of claim 9, wherein the physiological status is the pulse condition of the monitored individual.

16. The system of claim 9, wherein the physiological status is the heart beat condition of the monitored individual.

17. The system of claim 16, wherein the bio sensor is arranged on the wrist of the monitored individual for monitoring the pulse condition of the same.

18. The system of claim 9, wherein the bio sensor monitors the physiological status of the monitored individual by a non-contact means.
